(19) United States
(12) Patent Application Publication
(10) Pub. No.: US 2005/0187221 A1
(43) Pub. Date: Aug. 25, 2005

(54) Method of treating ischemia reperfusion injury

(75) Inventors: Hikaru Matsuda, Ashiya (JP); Yoshiki Sawa, Nishinomiya (JP); Masahiro Ryugo, Osaka (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Japan Tobacco Inc., Tokyo (JP)

(21) Appl. No.: 10/935,466

(22) Filed: Sep. 7, 2004

Related U.S. Application Data
(60) Provisional application No. 60/501,365, filed on Sep. 8, 2003.

(51) Int. Cl. A61K 31/495

(52) U.S. Cl. 514/252.12

(57) ABSTRACT

The invention provides a method of treating or preventing ischemia reperfusion injury in a subject in need thereof comprising administering a cytokine production inhibitor to a subject.

### CROSS-REFERENCE TO RELATED PATENT APPLICATIONS

This patent application claims the benefit of U.S. Provisional Patent Application No. 60/501,365 filed Sep. 8, 2003.

### FIELD OF THE INVENTION

This invention pertains to a method of treating or preventing ischemia reperfusion injury comprising administering a cytokine production inhibitor to a subject.

### BACKGROUND OF THE INVENTION

Recent advances in myocardial protection have improved the clinical results in open-heart surgery. Severely critical cases associated with compromised heart function, such as failing heart or postischemic conditions, still occur, and thus, further attempts to improve myocardial protection are needed.

Pro-inflammatory cytokines such as TNF-α, IL-1β, IL-6, and IL-8 have been shown to be induced by ischemia reperfusion, and to lead to myocardial dysfunction, directly or through the adherence of neutrophils to endothelial cells (see, e.g., Cain et al., Crit. Care Med., 27(7), 1309-1318 (1999); Kamikubo et al., Hokkaido Igakkai Zasshi, 68(6), 813-826 (1993); Tracey et al., Science, 234, 470-474 (1986); and Finkel et al., Science, 257, 387-389 (1992)). Therefore, several studies have been undertaken to attenuate cytokine-induced ischemia reperfusion injury (see, e.g., Kawamura et al., Crit. Care Med., 28, 2201-2208 (2000)). The clinical application of a few drugs has been attempted, but no compound has demonstrated the inhibition of the broad spectrum of inflammatory cytokines required for treatment of myocardial ischemia reperfusion injury. Therefore, there remains a need for a drug that inhibits multiple inhibitory cytokines for the treatment of ischemia reperfusion injury.

U.S. Pat. No. 6,174,887 and International Patent Application Publication WO 97/08133 A1 disclose amide compounds that exhibit superior suppressive effects on cytokines directly or indirectly involved in inflammations, such as interleukin (IL)-8, IL-1, IL-6, tumor necrosis factor (TNF)-α, and granulocyte-macrophage colony stimulating factor (GM-CSF), as well as methods of preparation thereof. The amide compounds are useful for the treatment and prophylaxis of inflammatory diseases. The inventors of the present invention unexpectedly discovered that the amide compounds of U.S. Pat. No. 6,174,887 and International Patent Application Publication WO 97/08133 A1 can be administered to a subject to treat or prevent ischemia reperfusion injury. These and other advantages of the invention, as well as additional inventive features, will be apparent from the description of the invention provided herein.

### BRIEF SUMMARY OF THE INVENTION

The invention provides a method of treating or preventing ischemia reperfusion injury in a subject in need thereof comprising administering a cytokine production inhibitor to a subject.

### BRIEF DESCRIPTION OF THE DRAWINGS

FIG. 1 is a plot of left ventricle developed pressure (LVDP) in Sprague-Dawley rats before and after ischemia reperfusion. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. *p<0.05.

FIG. 2 is a plot of the maximum rate of rise of left ventricular pressure (maximum dP/dt) in Sprague-Dawley rats before and after ischemia reperfusion. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. *p<0.05.

FIG. 3 is a plot of the percent recovery of left ventricle developed pressure (LVDP) over a 60-minute time period in Sprague-Dawley rats after ischemia reperfusion. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. Data are expressed as percentage of basal LVDP before ischemia. **p<0.01.

FIG. 4 is a plot of the percent recovery of the maximum rate of rise of left ventricular pressure (maximum dP/dt) over a 60-minute time period in Sprague-Dawley rats after ischemia reperfusion. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. Data are expressed as percentage of basal maximum dP/dt before ischemia. *p<0.05 and **p<0.01.

FIG. 5 is a plot of leakage of creatine phosphokinase (CPK) from cardiac myocytes into the coronary effluent in Sprague-Dawley rats after ischemia reperfusion. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. *p<0.05.

FIGS. 6A-6D are plots of the tissue level of inflammatory cytokines in the myocardium of Sprague-Dawley rats after ischemia reperfusion. FIG. 6A is a plot with respect to TNF-α, FIG. 6B is a plot with respect to IL-1β, FIG. 6C is a plot with respect to IL-6, and FIG. 6D is a plot with respect to IL-8. The C group signifies the control group, and the J group refers to rats that were administered (−)-ethyl N-{3,5-dichloro-2-hydroxy-4-[2-(4-methylpiperazin-1-yl)ethoxy]benzoyl}-L-phenylalaninate dihydrochloride. *p<0.05.

### DETAILED DESCRIPTION OF THE INVENTION

The invention provides a method of treating or preventing ischemia reperfusion injury in a subject in need thereof comprising administering a cytokine production inhibitor to a subject. Preferably, the cytokine production inhibitor is a compound of Formula (I): 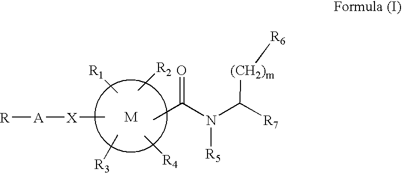
or a pharmaceutically acceptable acid addition salt thereof.

R is an optionally substituted non-aromatic heterocyclic group containing nitrogen, a hydroxy, Rₐ, an alkoxy substituted by Rₐ, an alkylthio substituted by Rₐ, or an alkylamino substituted by Rₐ. Rₐ is an amino, guanidino, amidino, carbamoyl, ureido, thioureido, hydrazino, hydrazinocarbonyl, or imino, wherein these groups optionally are substituted by a substituent selected from the group consisting of lower alkyl, halogenated lower alkyl, cycloalkyl, aralkyl, aryl, and amino-protecting group. Preferably, R is a piperazinyl optionally substituted by lower alkyl, a piperidyl optionally substituted by lower alkyl, or an amino optionally substituted by lower alkyl.

A is an optionally substituted, linear or branched alkylene which optionally has one or more double bond(s) or triple bond(s) in the chain, or a single bond. Preferably, A is a linear alkylene.

X is an oxygen atom, a sulfur atom, a cycloalkylene, a divalent aromatic heterocyclic group having one or more hetero atom(s) selected from the group consisting of a nitrogen atom, sulfur atom, and oxygen atom, —SO—, —SO₂—, —C═C—, —C≡C—, —CO—, —COO—, —OOC—, —CS—, —COS—, —O—CO—O—, —NH—CO—NH—, —NH—CS—NH—, —NH—C(═NH)—NH—, —NR₈—, —NR₈CO—, —CONR₈—, —NR₈SO₂—, —SO₂NR₈—, —NR₈—NR₈—, or —CR₉R₁₀—, wherein R₈ is a hydrogen atom, alkyl, cycloalkyl, aryl, aralkyl, or amino-protecting group, and R₉ and R₁₀ are the same or different and each is a hydrogen atom, alkyl, cycloalkyl, aryl, or aralkyl. Preferably, X is an oxygen atom, a sulfur atom, —NH—, or —CH₂—.

M is an arylene, a cycloalkylene, or a divalent heterocyclic group which has one or more hetero atom(s) selected from the group consisting of a nitrogen atom, sulfur atom, and oxygen atom, and which optionally forms a fused ring. Preferably, M is an arylene.

R₁, R₂, R₃, and R₄ are the same or different and each is a hydrogen atom, a hydroxy, a halogen atom, an alkoxy, a mercapto, an alkylthio, a nitro, a cyano, a carboxy, an alkoxycarbonyl, an aryloxycarbonyl, an acyl, an alkyl optionally substituted by a substituent selected from the group consisting of hydroxy, lower alkoxy, and halogen atom, amino optionally substituted by a substituent selected from the group consisting of alkyl, aryl, aralkyl, and amino-protecting group, or —O—CO—R₁₁. R₁₁ is an optionally substituted alkoxy, optionally substituted aryl, optionally substituted cycloalkyl, optionally substituted aryloxy, optionally substituted aralkyloxy, optionally substituted alkylthio, optionally substituted arylthio, or alkyl optionally substituted by a substituent selected from the group consisting of alkoxycarbonyl, acyloxy, aryl, aryloxy, aryloxycarbonyl, aralkyloxy, aralkyloxycarbonyl, alkylthio, arylthio, acyl, lower alkoxy, carboxy, halogen atom, and amino optionally substituted by lower alkyl or acyl. Preferably, R₁, R₂, R₃, and R₄ are the same or different and each is a hydrogen atom, provided at least one of R₁, R₂, R₃, and R₄ is not a hydrogen atom, a hydroxy, a halogen atom, or —O—CO—R₁₁, wherein R₁₁ is lower alkyl optionally substituted by a substituent selected from the group consisting of amino, acyloxy, and benzyloxycarbonyl, or phenyl optionally substituted by lower alkyl.

R₅ is a hydrogen atom, an alkyl optionally substituted by halogen atom, an optionally substituted aralkyl, or an amino-protecting group. Preferably R₅ is a hydrogen atom:
m is 0 or an integer of 1-6. Preferably m is 1.

R₆ is an optionally substituted aryl, an optionally substituted cycloalkyl, an optionally substituted lower alkyl, an optionally substituted lower alkoxy, an optionally substituted lower alkylthio, amino optionally substituted by a substituent selected from the group consisting of lower alkyl, aryl, aralkyl, and amino-protecting group, or an optionally substituted heterocyclic group having one or more hetero atoms selected from the group consisting of a nitrogen atom, sulfur atom, and oxygen atom. Preferably R₆ is a phenyl.

R₇ is a hydrogen atom, an optionally substituted alkyl, an optionally substituted aryl, an optionally substituted aromatic heterocyclic group having one or more hetero atoms selected from the group consisting of a nitrogen atom, sulfur atom, and oxygen atom, or —CO(Y)ₚR₁₂. Y is an oxygen atom, sulfur atom, —NR₁₃—, or —NR₁₃—SO₂—. R₁₃ is a hydrogen atom, alkyl, aralkyl, hydroxy, alkoxy, aryl, or amino-protecting group. p is 0 or 1. R₁₂ is a hydrogen atom, optionally substituted alkenyl, optionally substituted alkynyl, optionally substituted cycloalkyl, optionally substituted aryl, optionally substituted aralkyl, adamantyl, cycloalkylideneamino, optionally substituted heterocyclic group having one or more hetero atom(s) selected from the group consisting of nitrogen atom, sulfur atom, and oxygen atom, or alkyl optionally substituted by a substituent selected from the group consisting of hydroxy, alkoxy, alkoxyalkoxy, alkoxycarbonyl, acyloxy, carboxy, heterocyclic group having one or more hetero atom(s) selected from the group consisting of nitrogen atom, sulfur atom, and oxygen atom, and amino optionally substituted by a substituent selected from the group consisting of alkyl, aryl, aralkyl, and amino-protecting group. Preferably, R₇ is —COO—R₁₂, wherein R₁₂ is a hydrogen atom, aralkyl, adamantyl, cyclohexylideneamino, cyclohexyl optionally substituted by lower alkyl, piperidyl optionally substituted by lower alkyl, or alkyl optionally substituted by a substituent selected from the group consisting of hydroxy, lower alkoxy, lower alkoxy lower alkoxy, lower alkoxycarbonyl, acyloxy, piperazinyl, and amino optionally substituted by lower alkyl.

“Alkoxy” signifies a linear or branched alkoxy having 1 to 6 carbon atoms, which is exemplified by methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, and neohexyloxy, with preference given to a linear or branched alkoxy having 1 to 4 carbon atoms, such as methoxy, ethoxy, propoxy, isopropoxy, butoxy, isobutoxy, sec-butoxy, and tert-butoxy. The “alkoxy” of the substituted alkoxy at R preferably is a linear alkoxy, such as methoxy, ethoxy, propoxy, butoxy, pentyloxy, and hexyloxy, especially a linear alkoxy having 1 to 4 carbon atoms, such as methoxy, ethoxy, propoxy, and butoxy.

“Lower alkoxy” signifies a linear or branched alkoxy having 1 to 4 carbon atoms, which is exemplified by methoxy, ethoxy, propoxy, isopropoxy, butoxy, isobutoxy, sec-butoxy, and tert-butoxy, with preference given to methoxy and ethoxy.

“Alkylthio” signifies a linear or branched alkylthio having 1 to 6 carbon atoms, which is exemplified by methylthio, ethylthio, propylthio, isopropylthio, butylthio, isobutylthio, sec-butylthio, tert-butylthio, pentylthio, isopentylthio, neopentylthio, tert-pentylthio, hexylthio, isohexylthio, and neohexylthio. The “alkylthio” of the substituted alkylthio at R preferably is a linear alkylthio such as methylthio, ethylthio, propylthio, butylthio, pentylthio, and hexylthio, especially a linear alkylthio having 1 to 4 carbon atoms, such as methylthio, ethylthio, propylthio, and butylthio.

“Lower alkylthio” signifies a linear or branched alkylthio having 1 to 4 carbon atoms, which is exemplified by methylthio, ethylthio, propylthio, isopropylthio, butylthio, isobutylthio, sec-butylthio, and tert-butylthio.

“Alkylamino” signifies a linear or branched monoalkylamino or dialkylamino having 1 to 6 carbon atoms, which is exemplified by methylamino, dimethylamino, ethylamino, diethylamino, methylethylamino, propylamino, isopropylamino, butylamino, isobutylamino, sec-butylamino, tert-butylamino, pentylamino, isopentylamino, neopentylamino, tert-pentylamino, hexylamino, isohexylamino, and neohexylamino, with preference given to linear alkylamino, such as methylamino, dimethylamino, ethylamino, diethylamino, propylamino, butylamino, pentylamino, and hexylamino. Particularly preferred is a linear alkylamino having 1 to 4 carbon atoms, which is exemplified by methylamino, dimethylamino, ethylamino, diethylamino, propylamino, and butylamino.

“Non-aromatic heterocyclic group containing nitrogen” signifies a 3- to 7-membered non-aromatic heterocyclic group which has at least one nitrogen atom and optionally a sulfur atom or oxygen atom, and which is optionally fused with a benzene ring. Specific examples thereof include aziridinyl, thiazetidinyl, azetidinyl, pyrrolidinyl, pyrrolinyl, imidazolidinyl, imidazolinyl, pyrazolidinyl, pyrazolinyl, morpholinyl, morpholino, oxazinyl, thiazinyl, piperazinyl, piperidyl, piperidino, dioxazepinyl, thiazepinyl, diazepinyl, perhydrodiazepinyl, azepinyl, perhydroazepinyl, indolinyl, and isoindolinyl. Aziridinyl, azetidinyl, pyrrolidinyl, pyrazolidinyl, morpholinyl, morpholino, piperazinyl, piperidyl, piperidino, and perhydroazepinyl are preferred, with pyrrolidinyl, morpholino, piperazinyl, piperidyl, and piperidino being particularly preferred.

“Alkyl” signifies a linear or branched alkyl having 1 to 6 carbon atoms, which is exemplified by methyl, ethyl, propyl, isopropyl, butyl, isobutyl, sec-butyl, tert-butyl, pentyl, isopentyl, neopentyl, tert-pentyl, hexyl, isohexyl, and neohexyl.

“Lower alkyl” signifies a linear or branched alkyl having 1 to 4 carbon atoms, which is exemplified by methyl, ethyl, propyl, isopropyl, butyl, isobutyl, sec-butyl, and tert-butyl.

“Halogen atom” signifies a specifically a fluorine atom, chlorine atom, bromine atom, or iodine atom.

“Halogenated lower alkyl” signifies the above-mentioned lower alkyl substituted by a halogen atom. Suitable halogenated lower alkyls include fluoromethyl, chloromethyl, bromomethyl, difluoromethyl, dichloromethyl, trifluoromethyl, trichloromethyl, difluoroethyl, dichloroethyl, pentatrifluoroethyl, trichloroethyl, and fluoropropyl, with preference given to fluoromethyl, chloromethyl, difluoromethyl, dichloromethyl, and trifluoromethyl.

“Cycloalkyl” signifies a cyclic alkyl having 3 to 7 carbon atoms, which is exemplified by cyclopropyl, cyclobutyl, cyclopentyl, cyclohexyl, and cycloheptyl, with preference given to a cycloalkyl having 5 or 6 carbon atoms, such as cyclopentyl and cyclohexyl.

“Aralkyl” signifies an alkyl substituted by an aryl and is exemplified by benzyl, benzhydryl, trityl, phenethyl, 3-phenylpropyl, 2-phenylpropyl, 4-phenylbutyl, and naphthylmethyl, with preference given to benzyl and phenethyl.

“Aralkyloxy” signifies an aralkyloxy having the above-mentioned aralkyl and is exemplified by benzyloxy, benzhydryloxy, trityloxy, phenethyloxy, 3-phenylpropyloxy, 2-phenylpropyloxy, 4-phenylbutyloxy, and naphthylmethoxy, with preference given to benzyloxy and phenethyloxy.

“Aralkyloxycarbonyl” signifies an aralkyloxycarbonyl having the above-mentioned aralkyl and is exemplified by benzyloxycarbonyl, benzhydryloxycarbonyl, trityloxycarbonyl, phenethyloxycarbonyl, 3-phenylpropyloxycarbonyl, 2-phenylpropyloxycarbonyl, 4-phenylbutyloxycarbonyl, and naphthylmethoxy-carbonyl, with preference given to benzyloxycarbonyl and phenethyloxy-carbonyl.

“Aryl” signifies a phenyl, naphthyl, anthryl, phenanthryl, or biphenyl, with preference given to phenyl and naphthyl. “Aryloxy” is an aryloxy having the above-mentioned aryl and is exemplified by phenoxy and naphthyloxy.

“Aryloxycarbonyl” signifies an aryloxycarbonyl having the above-mentioned aryl and is exemplified by phenoxycarbonyl and naphthyloxycarbonyl.

“Arylthio” signifies an arylthio having the above-mentioned aryl and is exemplified by phenylthio and naphthylthio.

“Amino-protecting group” signifies a protecting group conventionally used, which is subject to no particular limitation as long as it protects the amino from various reactions. Specific examples include acyl (such as formyl, acetyl, propionyl, butyryl, oxalyl, succinyl, pivaloyl, 2-chloroacetyl, 2-bromoacetyl, 2-iodoacetyl, 2,2-dichloroacetyl, 2,2,2-trichloroacetyl, 2,2,2-trifluoroacetyl, phenylacetyl, phenoxyacetyl, benzoyl, 4-chlorobenzoyl, 4-methoxybenzoyl, 4-nitrobenzoyl, naphthyl-carbonyl, adamantylcarbonyl, and phthaloyl); alkoxycarbonyl (such as methoxycarbonyl, ethoxycarbonyl, propoxycarbonyl, isopropoxycarbonyl, tert-butoxycarbonyl, pentyloxycarbonyl, isopentyloxycarbonyl, cyclo-hexyloxycarbonyl, 2-chloroethoxycarbonyl, 2-iodoethoxycarbonyl, 2,2,2-trichloroethoxycarbonyl, 2,2,2-trichloro-tert-butoxycarbonyl, benzhydryloxycarbonyl, bis-(4-methoxyphenyl)methoxycarbonyl, phenacyloxy-carbonyl, 2-trimethylsilylethoxycarbonyl, 2-triphenylsilylethoxycarbonyl, and fluorenyl-9-methoxycarbonyl); alkenyloxycarbonyl (such as vinyloxy-carbonyl, 2-propenyloxycarbonyl, 2-chloro-2-propenyloxycarbonyl, 3-methoxycarbonyl-2-propenyloxycarbonyl, 2-methyl-2-propenyloxycarbonyl, 2-butenyloxycarbonyl, and cinnamyloxycarbonyl); aralkyloxycarbonyl (such as benzyloxycarbonyl, 4-bromobenzyloxycarbonyl, 2-chlorobenzyloxycarbonyl, 3-chlorobenzyloxycarbonyl, 3,5-dimethoxybenzyloxycarbonyl, 4-methoxy-benzyloxycarbonyl, 2-nitrobenzyloxycarbonyl, 4-nitrobenzyloxycarbonyl, 2-nitro-4,5-dimethoxybenzyloxycarbonyl, 3,4,5-trimethoxybenzyloxy-carbonyl, and phenethyloxycarbonyl); lower alkylsilyl (such as trimethyl-silyl and tert-butyldimethylsilyl); alkylenebis(dialkylsilyl) (such as ethylenebis(dimethylsilyl), propylenebis(dimethylsilyl), and ethylene-bis(diethylsilyl)); alkylthiocarbonyl (such as methylthiocarbonyl, ethylthiocarbonyl, butylthiocarbonyl, and tert-butylthiocarbonyl); aralkylthiocarbonyl (such as benzylthiocarbonyl); phosphoryl (such as dicyclohexylphosphoryl, diphenylphosphoryl, dibenzylphosphoryl, di-(4-nitrobenzyl)phosphoryl, and phenoxyphenylphosphoryl); and phosphinyl (such as diethylphosphinyl and diphenylphosphinyl).

“Linear or branched alkylene optionally having one or more double bond(s) or triple bond(s) in the chain” signifies a linear or branched alkylene having 1 to 10 carbon atoms, which may have one or more double bonds or triple bonds in the chain, and is exemplified by methylene, ethylene, trimethylene, tetramethylene, pentamethylene, hexamethylene, heptamethylene, octamethylene, nonamethylene, decamethylene, dimethylmethylene, diethylmethylene, propylene, methylethylene, ethylethylene, propylethylene, isopropylethylene, methylpentaethylene, ethylhexamethylene, dimethylethylene, methyltriethylene, dimethyltrimethylene, vinylene, propenylene, butenylene, butadienylene, pentenylene, pentadienylene, hexenylene, hexadienylene, hexatrienylene, heptenylene, heptadienylene, heptatrienylene, octenylene, octadienylene, octatrienylene, octatetraenylene, propynylene, butynylene, pentynylene, and methylpropynylene. Preferred is a linear alkylene, such as methylene, ethylene, trimethylene, tetramethylene, pentamethylene, hexamethylene, heptamethylene, octamethylene, nonamethylene, decamethylene, vinylene, propenylene, butenylene, butadienylene, pentenylene, pentadienylene, hexenylene, hexadienylene, hexatrienylene, heptenylene, heptadienylene, heptatrienylene, octenylene, octadienylene, octatrienylene, octatetraenylene, propynylene, butynylene, and pentynylene. Particularly preferred is a linear alkylene having 1 to 8 carbon atoms, such as methylene, ethylene, trimethylene, tetramethylene, pentamethylene, hexamethylene, heptamethylene, and octamethylene.

“Divalent aromatic heterocyclic group having one or more hetero atom(s) selected from the group consisting of a nitrogen atom, sulfur atom, and oxygen atom” is a 5- or 6-membered divalent aromatic heterocyclic group having one or more hetero atom(s) selected from the group consisting of a nitrogen atom, sulfur atom, and oxygen atom, which is exemplified by divalent groups of a tetrazole ring, oxadiazole ring, thiadiazole ring, triazole ring, oxazole ring, isoxazole ring, thiazole ring, isothiazole ring, imidazole ring, pyrazole ring, pyrrole ring, furan ring, thiophene ring, tetrazine ring, triazine ring, pyrazine ring, pyridazine ring, pyrimidine ring, and pyridine ring. Preferred is a 5-membered divalent aromatic heterocyclic group, which is exemplified by divalent groups of a tetrazole ring, oxadiazole ring, thiadiazole ring, triazole ring, oxazole ring, isoxazole ring, thiazole ring, isothiazole ring, imidazole ring, pyrazole ring, pyrrole ring, furan ring, and thiophene ring. Particularly preferred are divalent groups of an oxadiazole ring, thiadiazole ring, and triazole ring.

“Cycloalkylene” signifies a cycloalkylene having 3 to 7 carbon atoms, namely, divalent cycloalkyl, which is specifically exemplified by cyclopropylene, cyclobutylene, cyclopentylene, cyclohexylene, and cycloheptylene. Preferred is a cycloalkylene having 5 or 6 carbon atoms, which is exemplified by cyclopentylene and cyclohexylene.

“Arylene” is exemplified by phenylene, naphthylene, anthrylene, phenanthrylene, and biphenylene, with preference given to phenylene, naphthylene, and biphenylene.

“Divalent heterocyclic group which has one or more hetero atom(s) selected from the group consisting of a nitrogen atom, sulfur atom, and oxygen atom, and which optionally forms a fused ring” is specifically exemplified by divalent heterocyclic groups of a dioxolane ring, dithiol ring, pyrrolidine ring, morpholine ring, oxazine ring, piperazine ring, piperidine ring, pyrroline ring, imidazolidine ring, imidazoline ring, pyrazolidine ring, pyrazoline ring, thiatriazole ring, tetrazole ring, oxadiazole ring, thiadiazole ring, triazole ring, isoxazole ring, oxazole ring, thiazole ring, imidazole ring, pyrazole ring, pyrrole ring, furan ring, thiophene ring, tetrazine ring, triazine ring, pyrazine ring, pyridazine ring, pyrimidine ring, pyridine ring, furoisoxazole ring, imidazothiazole ring, thienoisothiazole ring, thienothiazole ring, imidazopyrazole ring, cyclopentapyrazole ring, pyrrolopyrrole ring, thienothiophene ring, thiadiazolopyrimidine ring, thiazolothiazine ring, thiazolopyrimidine ring, thiazolopyridine ring, oxazolopyrimidine ring, oxazolopyridine ring, benzoxazole ring, benzisothiazole ring, benzothiazole ring, imidazopyrazine ring, purine ring, pyrazolopyrimidine ring, imidazopyridine ring, benzimidazole ring, indazole ring, benzoxathiole ring, benzodioxole ring, benzodithiol ring, indolizine ring, indoline ring, isoindoline ring, furopyrimidine ring, furopyridine ring, benzofuran ring, isobenzofuran ring, thienopyrimidine ring, thienopyridine ring, benzothiophene ring, cyclopentaoxazine ring, cyclopentafuran ring, benzoxazine ring, benzothiazine ring, quinazoline ring, naphthyridine ring, quinoline ring, isoquinoline ring, benzopyran ring, pyridopyridazine ring, and pyridopyrimidine ring. Preferred are divalent heterocyclic groups of a piperazine ring, piperidine ring, pyridine ring, benzoxazole ring, benzisothiazole ring, benzothiazole ring, and benzimidazole ring.

“Alkoxycarbonyl” signifies a linear or branched alkoxycarbonyl having 2 to 7 carbon atoms, which is exemplified by methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, isopentyloxycarbonyl, neopentyloxycarbonyl, tert-pentyloxycarbonyl, hexyloxycarbonyl, isohexyloxycarbonyl, and neohexyloxycarbonyl, with preference given to a linear or branched alkoxycarbonyl having 2 to 5 carbon atoms, such as methoxycarbonyl, ethoxycarbonyl, propoxycarbonyl, isopropoxycarbonyl, butoxycarbonyl, isobutoxycarbonyl, sec-butoxycarbonyl, and tert-butoxycarbonyl.

“Lower alkoxycarbonyl” signifies a linear or branched alkoxycarbonyl having 2 to 5 carbon atoms, which is exemplified by methoxycarbonyl, ethoxycarbonyl, propoxycarbonyl, isopropoxycarbonyl, butoxycarbonyl, isobutoxycarbonyl, sec-butoxycarbonyl, and tert-butoxycarbonyl, with preference given to methoxycarbonyl and ethoxycarbonyl.

“Acyl” specifically means formyl, acetyl, propionyl, butyryl, isobutyryl, valeryl, isovaleryl, pivaloyl, caproyl, isocaproyl, acryloyl, propioloyl, methacryloyl, crotonoyl, isocrotonoyl, benzoyl, naphthoyl, toluoyl, hydroatropoyl, atropoyl, cinnamoyl, furoyl, glyceroyl, tropoyl, benziloyl, salicyloyl, anisoyl, vanilloyl, veratroyl, piperonyloyl, protocatechuoyl, or galloyl, with preference given to formyl, acetyl, propionyl, butyryl, isobutyryl, valeryl, isovaleryl, pivaloyl, benzoyl, and naphthoyl.

“Acyloxy” signifies an acyloxy having the above-mentioned acyl, which is exemplified by formyloxy, acetyloxy, propionyloxy, butyryloxy, isobutyryloxy, valeryloxy, isovaleryloxy, pivaloyloxy, caproyloxy, isocaproyloxy, acryloyloxy, propioloyloxy, methacryloyloxy, crotonoyloxy, isocrotonoyloxy, benzoyloxy, naphthoyloxy, toluoyloxy, hydroatropoyloxy, atropoyloxy, cinnamoyloxy, furoyloxy, glyceroyloxy, tropoyloxy, benziloyloxy, salicyloyloxy, anisoyloxy, vanilloyloxy, veratroyloxy, piperonyloyloxy, protocatechuoyloxy, and galloyloxy, with preference given to formyloxy, acetyloxy, propionyloxy, butyryloxy, isobutyryloxy, valeryloxy, isovaleryloxy, pivaloyloxy, benzoyloxy, and naphthoyloxy.

“Heterocyclic group having one or more hetero atom(s) selected from the group consisting of a nitrogen atom, sulfur atom, and oxygen atom” signifies a 3- to 7-membered heterocyclic group having one or more hetero atom(s) selected from the group consisting of a nitrogen atom, sulfur atom, and oxygen atom, which is exemplified by aziridinyl, oxiranyl, azetyl, azetidinyl, oxetanyl, thiatriazolyl, tetrazolyl, dithiazolyl, oxadiazolyl, thiadiazolyl, triazolyl, oxazolyl, isooxazolyl, thiazolyl, isothiazolyl, imidazolyl, pyrazolyl, dioxolanyl, pyrrolyl, pyrrolidinyl, furanyl, thienyl, tetrazinyl, dithiadiazinyl, thiadiazinyl, triazinyl, morpholinyl, morpholino, oxazinyl, thiazinyl, piperazinyl, pyrazinyl, pyridazinyl, pyrimidinyl, piperidyl, piperidino, pyridyl, pyranyl, thiopyranyl, dioxazepinyl, diazepinyl and azepinyl. Preferred is a 5- or 6-membered heterocyclic group, which is exemplified by thiatriazolyl, tetrazolyl, dithiazolyl, oxadiazolyl, thiadiazolyl, triazolyl, oxazolyl, isooxazolyl, thiazolyl, isothiazolyl, imidazolyl, pyrazolyl, dioxolanyl, pyrrolyl, pyrrolidinyl, furanyl, thienyl, tetrazinyl, dithiadiazinyl, thiadiazinyl, triazinyl, morpholinyl, morpholino, oxazinyl, thiazinyl, piperazinyl, pyrazinyl, pyridazinyl, pyrimidinyl, piperidyl, piperidino, pyridyl, pyranyl, and thiopyranyl. Particularly preferred at R₆ are pyrrolyl, furanyl, thienyl, piperazinyl, piperidyl, piperidino, and pyridyl. Particulary preferred at R₁₂ are pyrrolyl, piperazinyl, piperidyl, piperidino, and pyridyl.

“Aromatic heterocyclic group having one or more hetero atom(s) selected from the group consisting of a nitrogen atom, sulfur atom, and oxygen” signifies a 5- or 6-membered aromatic heterocyclic group having one or more hetero atom(s) selected from the group consisting of a nitrogen atom, sulfur atom, and oxygen atom, which is exemplified by tetrazolyl, oxadiazolyl, thiadiazolyl, triazolyl, oxazolyl, isooxazolyl, thiazolyl, isothiazolyl, imidazolyl, pyrazolyl, pyrrolyl, furanyl, thienyl, tetrazinyl, triazinyl, pyrazinyl, pyridazinyl, pyrimidinyl, and pyridyl. Preferred is a 5-membered aromatic heterocyclic group, which is exemplified by tetrazolyl, oxadiazolyl, thiadiazolyl, triazolyl, oxazolyl, isooxazolyl, thiazolyl, isothiazolyl, imidazolyl, pyrazolyl, pyrrolyl, furanyl, and thienyl. Particularly preferred are oxadiazolyl, thiadiazolyl, and triazolyl.

“Alkoxyalkoxy” signifies a linear or branched alkoxy having 1 to 6 carbon atoms substituted by a linear or branched alkoxy having 1 to 6 carbon atoms, and is exemplified by methoxymethoxy, ethoxymethoxy, propoxymethoxy, isopropoxymethoxy, butoxymethoxy, iso-butoxymethoxy, sec-butoxymethoxy, tert-butoxymethoxy, pentyloxymethoxy, isopentyloxymethoxy, neopentyloxymethoxy, tert-pentyloxymethoxy, hexyloxymethoxy, isohexyloxymethoxy, neohexyloxymethoxy, tert-hexyl-oxymmthoxy, methoxyethoxy, ethoxyethoxy, propoxyethoxy, isopropoxy-ethoxy, butoxyethoxy, isobutoxyethoxy, sec-butoxyethoxy, tert-butoxy-ethoxy, pentyloxyethoxy, isopentyloxyethoxy, neopentyloxyethoxy, tert-pentyloxyethoxy, hexyloxyethoxy, isohexyloxyethoxy, neohexyloxyethoxy, tert-hexyloxyethoxy, methoxypropoxy, ethoxypropoxy, propoxypropoxy, isopropoxypropoxy, butoxypropoxy, isobutoxypropoxy, sec-butoxypropoxy, tert-butoxypropoxy, pentyloxypropoxy, isopentyloxypropoxy, neopentyl-oxypropoxy, tert-pentyloxypropoxy, hexyloxypropoxy, isohexyloxypropoxy, neohexyloxypropoxy, tert-hexyloxypropoxy, methoxybutoxy, ethoxybutoxy, propoxybutoxy, isopropoxybutoxy, butoxybutoxy, isobutoxybutoxy, sec-butoxybutoxy, tert-butoxybutoxy, pentyloxybutoxy, isopentyloxybutoxy, neopentyloxybutoxy, tert-pentyloxybutoxy, hexyloxybutoxy, isohexyloxy-butoxy, neohexyloxybutoxy, tert-hexyloxybutoxy, methoxypentyloxy, ethoxypentyloxy, propoxypentyloxy, isopropoxypentyloxy, butoxypentyloxy, isobutoxypentyloxy, sec-butoxypentyloxy, tert-butoxypentyloxy, pentyloxypentyloxy, isopentyloxypentyloxy, neopentyloxypentyloxy, tert-pentyloxypentyloxy, hexyloxypentyloxy, isohexyloxypentyloxy, neo-hexyloxypentyloxy, tert-hexyloxypentyloxy, methoxyhexyloxy, ethoxy-hexyloxy, propoxyhexyloxy, isopropoxyhexyloxy, butoxyhexyloxy, iso-butoxyhexyloxy, sec-butoxyhexyloxy, tert-butoxyhexyloxy, pentyloxy-hexyloxy, isopentyloxyhexyloxy, neopentyloxyhexyloxy, tert-pentyloxy-hexyloxy, hexyloxyhexyloxy, isohexyloxyhexyloxy, neohexyloxyhexyloxy, and tert-hexyloxyhexyloxy. Preferred is a linear or branched alkoxy having 1 to 4 carbon atoms substituted by a linear or branched alkoxy having 1 to 4 carbon atoms, which is exemplified by methoxymethoxy, ethoxymethoxy, propoxymethoxy, isopropoxymethoxy, butoxymethoxy, isobutoxymethoxy, sec-butoxymethoxy, tert-butoxymethoxy, methoxyethoxy, ethoxyethoxy, propoxyethoxy, isopropoxyethoxy, butoxy-ethoxy, isobutoxyethoxy, sec-butoxyethoxy, tert-butoxyethoxy, methoxy-propoxy, ethoxypropoxy, propoxypropoxy, isopropoxypropoxy, butoxy-propoxy, isobutoxypropoxy, sec-butoxypropoxy, tert-butoxypropoxy, methoxybutoxy, ethoxybutoxy, propoxybutoxy, isopropoxybutoxy, butoxybutoxy, isobutoxybutoxy, sec-butoxybutoxy, and tert-butoxybutoxy.

“Lower alkoxy lower alkoxy” signifies a linear or branched alkoxy having 1 to 4 carbon atoms substituted by a linear or branched alkoxy having 1 to 4 carbon atoms, and is exemplified by methoxymethoxy, ethoxymethoxy, propoxymethoxy, isopropoxymethoxy, butoxymethoxy, isobutoxymethoxy, sec-butoxymethoxy, tert-butoxymethoxy, methoxyethoxy, ethoxyethoxy, propoxyethoxy, isopropoxyethoxy, butoxyethoxy, isobutoxyethoxy, sec-butoxyethoxy, tert-butoxyethoxy, methoxypropoxy, ethoxypropoxy, propoxypropoxy, isopropoxypropoxy, butoxypropoxy, isobutoxypropoxy, sec-butoxypropoxy, tert-butoxypropoxy, methoxybutoxy, ethoxybutoxy, propoxybutoxy, isopropoxybutoxy, butoxybutoxy, isobutoxybutoxy, sec-butoxybutoxy, and tert-butoxybutoxy, with preference given to methoxymethoxy, ethoxymethoxy, methoxyethoxy, and ethoxyethoxy.

“Alkenyl” signifies a linear or branched alkenyl having 2 to 6 carbon atoms, which is exemplified by allyl, vinyl, propenyl, isopropenyl, 1-methyl-2-propenyl, 2-methyl-2-propenyl, 1-methyl-1-butenyl, crotyl, 1-methyl-3-butenyl, 3-methyl-2-butenyl, 1-pentenyl, 1-methyl-2-pentenyl, 4-pentenyl, 1-hexenyl, 3-hexenyl, and 4-hexenyl.

“Alkynyl” signifies a linear or branched alkynyl having 2 to 6 carbon atoms, which is exemplified by propargyl, 2-butynyl, 1-methyl-2-butynyl, 2-pentynyl, 1-methyl-3-pentynyl, 1-methyl-4-pentynyl, 1-hexynyl, and 5-hexynyl.

“Cycloalkylideneamino” specifically means cyclopropylideneamino, cyclobutylideneamino, cyclopentylideneamino, cyclohexylideneamino, and cycloheptylideneamino, with preference given to cyclopentylideneamino and cyclohexylideneamino.

“Optionally substituted” of “optionally substituted non-aromatic heterocyclic group containing nitrogen” means that the group may be substituted by 1 to 3 substituent(s), and the substituents may be the same or different. The position of the substituent(s) is optional and is not particularly limited. Specific examples of the substituents include the above-mentioned lower alkyl, the above-mentioned halogenated lower alkyl, the above-mentioned cycloalkyl, the above-mentioned aralkyl, the above-mentioned aryl, and the above-mentioned amino-protecting group. Preferred are a lower alkyl and amino-protecting group.

“Optionally substituted” of “optionally substituted linear or branched alkylene which may have one or more double bond(s) or triple bond(s) in the chain” means that the group may be substituted by one or more substituent(s), and the substituents may be the same or different. Examples of the substituents include the above-mentioned halogen atom, hydroxy, amino (which may be substituted by a substituent selected from the group consisting of the above-mentioned lower alkyl, the above-mentioned halogenated lower alkyl, the above-mentioned cycloalkyl, the above-mentioned aralkyl, the above-mentioned aryl, and the above-mentioned amino-protecting group), the above-mentioned lower alkoxy, the above-mentioned aralkyl, and the above-mentioned cycloalkyl.

“Optionally substituted” of “optionally substituted alkoxy,” and “optionally substituted alkylthio” at R₁₁ means that the group may be substituted by one or more substituent(s), and the substituents may be the same or different. The position of the substituent(s) is optional and is not particularly limited. Specific examples of the substituents include the above-mentioned halogen atom, the above-mentioned lower alkoxy, the above-mentioned alkylthio, amino (which may be substituted by the above-mentioned lower alkyl or the above-mentioned acyl), carboxy, the above-mentioned alkoxycarbonyl, the above-mentioned acyl, the above-mentioned acyloxy, the above-mentioned aryl, the above-mentioned aryloxy, the above-mentioned arylthio, the above-mentioned aryloxycarbonyl, the above-mentioned aralkyloxy, and the above-mentioned aralkyloxycarbonyl. Preferred are an amino, lower alkoxy, halogen atom, carboxy, alkoxycarbonyl, and aralkyloxycarbonyl.

“Optionally substituted” of “optionally substituted aryl,” “optionally substituted cycloalkyl,” “optionally substituted aryloxy,” “optionally substituted aralkyloxy,” and “optionally substituted arylthio” at R₁₁ means that the group may have 1 to 3 substituent(s) on the ring, and the substituents may be the same or different. The position of the substituent(s) is optional and is not particularly limited. Specific examples of the substituents include the above-mentioned lower alkyl, the above-mentioned halogen atom, the above-mentioned lower alkoxy, the above-mentioned alkylthio, amino(which may be substituted by the above-mentioned lower alkyl or the above-mentioned acyl), carboxy, the above-mentioned alkoxycarbonyl, the above-mentioned acyl, the above-mentioned acyloxy, the above-mentioned aryl, the above-mentioned aryloxy, the above-mentioned arylthio, the above-mentioned aryloxycarbonyl, the above-mentioned aralkyloxy, and the above-mentioned aralkyloxycarbonyl. Preferred are lower alkyl, amino, lower alkoxy, halogen atom, carboxy, alkoxycarbonyl and aralkyloxycarbonyl. Particularly preferred is a lower alkyl.

“Optionally substituted” of “optionally substituted aralkyl” at R₅ means that it may have 1 to 3 substituent(s) on the aryl, and the substituents may be the same or different. The position of the substituent(s) is optional and is not particularly limited. Specific examples of the substituents include the above-mentioned lower alkyl, the above-mentioned lower alkoxy, the above-mentioned acyl, amino (which may be substituted by the above-mentioned lower alkyl or the above-mentioned acyl), the above-mentioned alkoxycarbonyl, the above-mentioned aryloxycarbonyl, the above-mentioned aryloxy, the above-mentioned alkylthio, the above-mentioned arylthio, the above-mentioned aryl, and the above-mentioned halogen atom. Preferred are lower alkyl, lower alkoxy, and halogen atom. Particularly preferred is lower alkyl.

“Optionally substituted” of “optionally substituted lower alkyl,” “optionally substituted lower alkoxy,” and “optionally substituted lower alkylthio” at R₆ means that the group may be substituted by one or more substituent(s), and the substituents may be the same or different. The position of the substituent(s) is optional and is not particularly limited. Specific examples of the substituents include the above-mentioned halogen atom, hydroxy, the above-mentioned alkoxy, the above-mentioned aryloxy, amino (which may be substituted by the above-mentioned lower alkyl or the above-mentioned acyl), mercapto, the above-mentioned alkylthio, the above-mentioned arylthio, carboxy, the above-mentioned alkoxycarbonyl, the above-mentioned aryloxycarbonyl, carbamoyl, the above-mentioned halogenated lower alkyl, sulfamoyl, cyano, nitro, alkylsulfonyl (such as methylsulfonyl, ethylsulfonyl, and isopropylsulfonyl), alkylsulfinyl (such as methylsulfinyl, ethylsulfinyl, and isopropylsulfinyl), and arylsulfonyl (such as phenylsulfonyl). Preferred are a halogen atom, hydroxy, alkoxy, amino, carboxy, and alkoxycarbonyl.

“Optionally substituted” of “optionally substituted aryl,” “optionally substituted cycloalkyl,” and “optionally substituted heterocyclic group having one or more hetero atom(s) selected from the group consisting of a nitrogen atom, sulfur atom, and oxygen atom” at R₆ means that the group may be substituted by one or more substituent(s), and the substituents may be the same or different. The position of the substituent(s) is optional and is not particularly limited. Specific examples of the substituents include the above-mentioned lower alkyl, the above-mentioned halogen atom, hydroxy, the above-mentioned alkoxy, the above-mentioned aryloxy, amino (which may be substituted by the above-mentioned lower alkyl or the above-mentioned acyl), mercapto, the above-mentioned alkylthio, the above-mentioned arylthio, carboxy, the above-mentioned alkoxycarbonyl, the above-mentioned aryloxycarbonyl, carbamoyl, the above-mentioned halogenated lower alkyl, sulfamoyl, cyano, nitro, alkylsulfonyl (such as methylsulfonyl, ethylsulfonyl, and isopropylsulfonyl), alkylsulfinyl (such as methylsulfinyl, ethylsulfinyl, and isopropylsulfinyl), and arylsulfonyl (such as phenylsulfonyl). Preferred are lower alkyl, halogen atom, hydroxy, alkoxy, amino, carboxy, and alkoxycarbonyl.

“Optionally substituted” of “optionally substituted alkyl” at R₇ means that the group may be substituted by one or more substituent(s), and the substituent(s) may be the same or different. The position of the substituent(s) is optional and is not particularly limited. Specific examples of the substituents include hydroxy, the above-mentioned lower alkoxy, mercapto, the above-mentioned lower alkylthio, carboxy, the above-mentioned lower alkoxycarbonyl, halogen atom, and amino which may be substituted by the above-mentioned lower alkyl or the above-mentioned acyl. Preferred are hydroxy, halogen atom, and lower alkoxy.

“Optionally substituted” of “optionally substituted aryl” and “optionally substituted aromatic heterocyclic group having one or more hetero atom(s) selected from the group consisting of a nitrogen atom, sulfur atom, and oxygen atom” at R₇ means that the group may have 1 to 3 substituent(s) on the ring, and the substituents may be the same or different. The position of the substituent(s) is optional and is not particularly limited. Specific examples of the substituents include the above-mentioned lower alkyl, hydroxy, the above-mentioned lower alkoxy, mercapto, the above-mentioned lower alkylthio, carboxy, the above-mentioned lower alkoxycarbonyl, halogen atom, and amino (which may be substituted by the above-mentioned lower alkyl or the above-mentioned acyl). Preferred are hydroxy, lower alkyl, halogen atom, and lower alkoxy.

“Optionally substituted” of “optionally substituted alkenyl” and “optionally substituted alkynyl” at R₁₂ means that the group may be substituted by one or more substituent(s), and the substituent(s) may be the same or different. The position of the substituent(s) is optional and is not particularly limited. Specific examples of the substituents include hydroxy, the above-mentioned alkoxy, carboxy, the above-mentioned alkoxycarbonyl, the above-mentioned acyloxy, amino (which may be substituted by the above-mentioned alkyl), the above-mentioned aryl, the above-mentioned aralkyl, or the above-mentioned amino-protecting group. Preferred are hydroxy, alkoxy, carboxy, alkoxycarbonyl, and acyloxy.

“Optionally substituted” of “optionally substituted cycloalkyl,” “optionally substituted aryl,” and “optionally substituted heterocyclic group having one or more hetero atom(s) selected from the group consisting of a nitrogen atom, sulfur atom, and oxygen atom” at R₁₂ means that the group may have 1 to 3 substituent(s) on the ring, and the substituents may be the same or different. The position of the substituent(s) is optional and is not particularly limited. Specific examples of the substituents include hydroxy, the above-mentioned lower alkoxy, mercapto, the above-mentioned lower alkylthio, carboxy, the above-mentioned lower alkoxycarbonyl, the above-mentioned lower alkyl, amino (which may be substituted by the above-mentioned lower alkyl), the above-mentioned halogen atom, carbamoyl, cyano, the above-mentioned acyl, nitro, sulfamoyl, alkoxythiocarbonyl, thioalkanoyl, alkylsulfonyl (such as methylsulfonyl and ethylsulfonyl), azomethine (which may be substituted by the above-mentioned lower alkyl, the above-mentioned aryl, or the above-mentioned aralkyl), alkoxyamino (such as methoxyamino and isopropoxyamino), hydrazino (which may be substituted by the above-mentioned lower alkyl, the above-mentioned aryl, or the above-mentioned aralkyl), aminooxy (which may be substituted by the above-mentioned lower alkyl, the above-mentioned aryl, or the above-mentioned aralkyl), and alkylsulfinyl (such as methylsulfinyl). Preferred are hydroxy, lower alkyl, halogen atom, lower alkoxy, amino, and carboxy.

“Optionally substituted” of “optionally substituted aralkyl” at R₁₂ means that the group may have 1 to 3 substituent(s) on the aryl, and the substituents may be the same or different. The position of the substituent(s) is optional and is not particularly limited. Specific examples of the substituents include the above-mentioned lower alkyl, the above-mentioned lower alkoxy, the above-mentioned acyl, amino (which may be substituted by the above-mentioned lower alkyl or the above-mentioned acyl), the above-mentioned alkoxycarbonyl, the above-mentioned aryloxycarbonyl, the above-mentioned aryloxy, the above-mentioned alkylthio, the above-mentioned arylthio, the above-mentioned aryl, and the above-mentioned halogen atom. Preferred are lower alkyl, lower alkoxy, and halogen atom.

Acid addition salts suitable for use in the invention include acid addition salts formed from inorganic acids (e.g., hydrochloric acid, sulfuric acid, phosphoric acid, hydrobromic acid, and nitric acid) and organic acids (e.g., oxalic acid, maleic acid, fumaric acid, malic acid, tartaric acid, succinic acid, citric acid, acetic acid, lactic acid, methanesulfonic acid, p-toluenesulfonic acid, benzoic acid, valeric acid, malonic acid, nicotinic acid, and propionic acid). Preferred acid addition salts of the invention are those formed from hydrochloric acid. Preferably, the acid addition salt of the invention is dihydrochloride.

The cytokine production inhibitors to be used in the method of the invention also include one or more stereoisomers due to an asymmetric carbon atom. Such isomers and mixtures thereof are encompassed by the invention. In addition, hydrates and solvates with pharmaceutically acceptable organic solvents, as well as prodrugs of the cytokine production inhibitors, are encompassed by the invention.

Preferably, the cytokine production inhibitor is (−)-ethyl N-{3,5-dichloro-2-hydroxy-4-[2-(4-methylpiperazin-1-yl)ethoxy]benzoyl}-L-phenylalaninate (i.e., N-{3,5-dichloro-2-hydroxy-4-[2-(4-methylpiperazin-1-yl)ethoxy]benzoyl}-L-phenylalanine ethyl ester), which is represented by Formula (II): 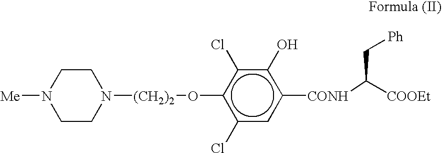
or a pharmaceutically acceptable acid addition salt thereof.

More preferably, the cytokine production inhibitor is (−)-ethyl N-{3,5-dichloro-2-hydroxy-4-[2-(4-methylpiperazin-1-yl)ethoxy]benzoyl}-L-phenylalaninate dihydrochloride (i.e., N-{3,5-dichloro-2-hydroxy-4-[2-(4-methylpiperazin-1-yl)ethoxy]-benzoyl}-L-phenylalanine ethyl ester dihydrochloride), which is represented by Formula (II-a):

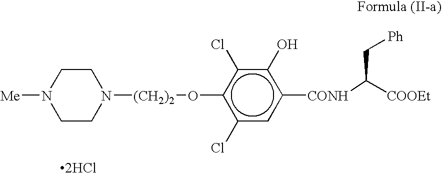

The cytokine production inhibitor may be administered for therapy to a subject in any conventional manner. While it is possible for the cytokine production inhibitor to be administered as the raw chemical, it is preferably administered as a pharmaceutical composition. Such a pharmaceutical composition comprises the cytokine production inhibitor with one or more pharmaceutically acceptable carriers or excipients and optionally other therapeutic agents and/or components. The carriers or excipients must be acceptable in the sense of being compatible with the other ingredients and not deleterious to the recipient thereof.

When the cytokine production inhibitor is used in a pharmaceutical composition comprising the same as an active ingredient, it is generally admixed with a pharmaceutically acceptable carrier, excipient, diluent, extender, disintegrator, stabilizer, preservative, buffer, emulsifying agent, aromatic agent, coloring agent, sweetener, thickener, flavor, solubilizer, and/or other additive such as water, vegetable oil, alcohol (e.g., ethanol and benzyl alcohol), polyethylene glycol, glycerol triacetate, gelatin, lactose, carbohydrate (e.g., starch), magnesium stearate, talc, lanolin, and/or white petrolatum known to give a pharmaceutical composition in the form of tablet, pill, powder, granule, suppository, injection, eye drop, liquid, capsule, troche, aerosol, elixir, suspension, emulsion, syrup, or the like. For oral administration, fine powders or granules may contain diluting, dispersing, and/or surface active agents and may be present, for example, in water or in a syrup, in capsules or sachets in the dry state, in a nonaqueous solution or suspension wherein suspending agents may be included, or in tablets wherein binders and lubricants may be included. Components such as sweeteners, flavoring agents, preservatives (e.g., antimicrobial preservatives), suspending agents, thickening agents, and/or emulsifying agents also may be present in the pharmaceutical composition. For parenteral administration, the pharmaceutical composition is in the form of a liquid solution or suspension and can contain the cytokine production inhibitor and purified water or saline. Optional components in the liquid solution or suspension include preservatives (e.g., antimicrobial preservatives), buffering agents, solvents, and mixtures thereof. A component of the pharmaceutical composition may serve more than one function. The pharmaceutical composition may be presented in unit-dose or multi-dose containers, for example, sealed vials and ampoules, and may be stored in a freeze-dried (lyophilized) condition requiring only the addition of the sterile liquid carrier, for example, water or saline, prior to use. In a preferred embodiment, the cytokine production inhibitor is formulated as a freeze-dried preparation containing the inhibitor and D-mannitol. The freeze-dried preparation is preferably diluted with saline prior to use.

The pharmaceutical composition comprising the cytokine production inhibitor can be prepared by any suitable method, such as those methods well known in the art of pharmacy, for example, the methods described in Gennaro et al., Remington's Pharmaceutical Sciences (18th ed., Mack Publishing Co., 1990), especially Part 8: Pharmaceutical Preparations and their Manufacture. Such methods include the step of bringing into association the cytokine production inhibitor with the other components of the pharmaceutical composition.

The cytokine production inhibitor may be administered to a subject in any suitable manner. The routes of administration include, but are not limited to, oral, buccal, nasal, transdermal, injectable, slow release, controlled release, iontophoresis, and sonophoresis. Injectable methods include, but are not limited to, parenteral routes of administration, such as intravenous, intramuscular, subcutaneous, intraperitoneal, intraspinal, intrathecal, intracerebroventricular, intraarterial, and other routes of injection. For parenteral administration, the pharmaceutical composition can be an aqueous or non-aqueous sterile injection formulation. Preferably, the cytokine production inhibitor is administered by injection (e.g., intraveneously).

The suitable dose of the cytokine production inhibitor varies depending on the type and severity of ischemia reperfusion injury, the administration route, age, sex, body weight, and the like of the subject. When orally administered to an adult human, for example, the daily dose of the cytokine production inhibitor (especially the compound of Formula (II) or (II-a)) is generally about 0.01-1,000 mg (e.g., about 0.05 mg, about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, or ranges thereof), and preferably about 0.1-100 mg. When the cytokine production inhibitor (especially the compound of Formula (II) or (II-a)) is administered intraveneously to an adult human, for example, the daily dose is generally about 0.01-100 mg/kg (e.g., about 0.05 mg/kg, about 0.1 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or ranges thereof), and preferably about 0.01-50 mg/kg, which is administered in one or several doses (e.g., two, three, four, five, six, seven, eight, nine, ten, or more). Alternatively, the cytokine production inhibitor (especially the compound of Formula (II) or (II-a)) may be administered by continuous intraveneous administration over a selected period of time (e.g., from several hours to one or more days). The total daily dose from continuous administration will generally be the same as the daily doses utilized for non-continuous intraveneous administration.

The cytokine production inhibitor can be administered to the subject at any suitable time to treat or prevent ischemia reperfusion injury. For example, the cytokine production inhibitor can be administered from about 2 hours before reperfusion to about 30 minutes following reperfusion (e.g., about 1.75 hours before reperfusion, about 1.5 hours before reperfusion, about 1.25 hours before reperfusion, about 1 hour before reperfusion, about 50 minutes before reperfusion, about 40 minutes before reperfusion, about 30 minutes before reperfusion, about 20 minutes before reperfusion, about 10 minutes before reperfusion, approximately at the same time as reperfusion, about 5 minutes after reperfusion, about 10 minutes after reperfusion, about 15 minutes after reperfusion, about 20 minutes after reperfusion, about 25 minutes after reperfusion, or ranges thereof).

Ischemia reperfusion injury refers to an injury which occurs after blood circulation is restarted in an organic tissue fallen into ischemia (e.g., when an excision operation or ablation of various organs is conducted). Such injury also occurs when blood circulation is restarted after being stopped for the transplantation of an organ; after the coronary artery is treated with percutaneous transluminal coronary angioplasty (PTCA), stent, or bypass after myocardial infarction; and after administration of a thrombolytic to a stroke patient. Such an injury can occur (and frequently does occur) in many tissues, such as kidney, liver, lungs, pancreas, skeletal muscle, and intestines, as well as in the heart and brain. The ischemia reperfusion injury to be treated or prevented by the method of the invention includes, but is not limited to, cerebral, retinal, hepatic, renal, spinal cord, mesenteric, limb, intestinal, brain, myocardial, central nervous system, or lung ischemia reperfusion injury, or a combination thereof. Preferably, the ischemia reperfusion injury is myocardial ischemia reperfusion injury.

A “subject” refers to an animal, preferably a mammal. For example, mammals include, but are not limited to, rats, mice, pigs, cats, dogs, cows, horses, monkeys, and humans. Preferably, the mammal is a human (male or female).

While not wishing to be bound by any particular theory, it is believed that the cytokine production inhibitor to be used in the method of the invention inhibits ischemia reperfusion injury in a subject by inhibiting pro-inflammatory cytokines. Specifically, the cytokine production inhibitor to be used in the method of the invention may directly inhibit the upper portion of the signaling pathway of inflammatory production.

Previous studies have shown that cytokines are elevated after ischemia reperfusion in animals, including humans, and that pro-inflammatory cytokines have direct effects on the depression of myocardial contractility. For example, IL-1β, IL-6, IL-8, interferon (IFN)-γ, and TNF-α have been shown to be upregulated following global ischemia. Therefore, the invention is directed to a method of treating or preventing ischemia reperfusion injury comprising the administration of the cytokine production inhibitor, wherein the production of one or more (e.g., two, three, four, five, or more) pro-inflammatory cytokines is inhibited. Preferably, the inhibited cytokine is TNF-α, IL-1β, IL-6, IL-8, IL-10, or granulocyte-macrophage colony stimulating factor (GM-CSF). More preferably, the inhibited cytokine is IL-6, IL-8, TNF-α, or a combination thereof. Most preferably, the cytokine production inhibitor inhibits the production of two or more pro-inflammatory cytokines (e.g., three or more, four or more, or five or more), such that the cytokine production inhibitor can be considered to have a broad effect for suppressing pro-inflammatory cytokines.

Additionally, the invention is directed to a method of treating or preventing ischemia reperfusion injury comprising the administration of the cytokine production inhibitor, wherein after the administration of the cytokine production inhibitor, the amount of creatine phosphokinase (CPK) released from a muscle after ischemia reperfusion is less than the amount of CPK released when the compound is not administered to the subject. CPK is an enzyme present in skeletal muscle, smooth muscle, and cardiac muscle that is released into the bloodstream in increased quantities if muscle is injured. Thus, lower levels of CPK in the cardiac effluent following the administration of the cytokine production inhibitor and ischemia reperfusion as compared to the levels observed without administration of the cytokine production inhibitor indicates treatment or prophylaxis of ischemia reperfusion following preconditioning with the cytokine production inhibitor.

Moreover, the invention is directed to a method of treating or preventing ischemia reperfusion injury in a subject in need thereof comprising administering the cytokine production inhibitor, wherein the percent recovery of LVDP is greater than the percent recovery of LVDP in the subject when the cytokine production inhibitor is not administered. For example, the percent recovery of LVDP in the subject administered the cytokine production inhibitor is greater than the percent recovery without the administration of the cytokine production inhibitor by about 5% or more (e.g., about 10% or more, about 15% or more, about 20% or more about 25% or more, about 30% or more, about 35% or more, about 40% or more, about 45% or more, about 50% or more, or about 55% or more).

Similarly, the invention is directed to a method of treating or preventing ischemia reperfusion injury in a subject in need thereof comprising administering the cytokine production inhibitor, wherein the percent recovery of maximum rate of rise of left ventricular pressure (maximum dP/dt) is greater than the percent recovery of maximum dP/dt in the subject when the cytokine production inhibitor is not administered. For example, the percent recovery of maximum dP/dt in the subject administered the cytokine production inhibitor is greater than the percent recovery without the administration of the cytokine production inhibitor by about 5% or more (e.g., about 10% or more, about 15% or more, about 20% or more about 25% or more, about 30% or more, about 35% or more, about 40% or more, or about 45% or more).

### EXAMPLE

This example further illustrates the invention but, of course, should not be construed as in any way limiting its scope. This example demonstrates the cardioprotective effect of preconditioning with the cytokine production inhibitor (−)-ethyl N-{3,5-dichloro-2-hydroxy-4-[2-(4-methylpiperazin-1-yl)ethoxy]benzoyl}-L-phenylalaninate dihydrochloride.

Sixteen Sprague-Dawley rats (300 g, male) were divided into a control group (“C group,” n=8) and a group administered (−)-ethyl N-{3,5-dichloro-2-hydroxy-4-[2-(4-methylpiperazin-1-yl)ethoxy]benzoyl}-L-phenylalaninate dihydrochloride (“J group,” n=8). All rats were anesthesized by intraperitoneal injection of sodium pentobarbital (50 mg/kg), and either (a) 0.5 mL of saline (C group) or (b) 10 mg/kg of (−)-ethyl N-{3,5-dichloro-2-hydroxy-4-[2-(4-methylpiperazin-1-yl)ethoxy]benzoyl}-L-phenylalaninate dihydrochloride dissolved in 5% mannitol (J group) was administered intraperitoneally to the rats. Anticoagulation was ensured by the intraperitoneal administration of 200 USP units of heparin.

Ten minutes after the administration of heparin and either (a) saline (C group) or (b) 10 mg/kg of (−)-ethyl N-{3,5-dichloro-2-hydroxy-4-[2-(4-methylpiperazin-1-yl)ethoxy]benzoyl}-L-phenylalaninate dihydrochloride dissolved in 5% mannitol (J group), the hearts of the rats were excised and perfused with modified Krebs-Henseleit buffer (120.0 mM NaCl, 4.5 mM KCl, 20.0 mM NaHCO₃, 1.2 mM KH₂PO₄, 1.2 MgCl₂, 2.5 mM CaCl₂, and 10.0 mM glucose: gassed with 95% O₂+5% CO₂ to obtain a pH of 7.4 at 37° C.) at the pressure equal to 1 m H₂O by means of a Langendorff apparatus. A thin-wall latex balloon was inserted into the left ventricle through the left atrium to monitor left ventricular pressure and to control left ventricular volume.

After stabilization, heart rate (HR), left ventricular developed pressure (LVDP), maximum dP/dt (maximum dP/dt), and coronary flow (CF) were measured with LV diastolic pressure stabilized at 10 mm Hg. The hearts were subjected to global ischemia at 37° C. for 30 minutes, followed by 60 minutes of reperfusion. The balloon was deflated during ischemia, and the hearts were not paced during reperfusion. HR, LVDP, maximum dP/dt, and CF were continuously measured after reperfusion and analyzed using a Polygraph System (Nihon Kouden, Japan).

Additionally, the coronary effluent was collected in chilled vials to measure creatine phosphokinase (CPK) levels after reperfusion. CPK can be fractionated so that specific measurements can be made for blood levels of CPK that comes exclusively from damaged heart muscle.

After 60 minutes of reperfusion, frozen sections of the hearts were made and stored at −80° C. for further assessment. The frozen tissue samples were homogenized with a Polytron homogenizer (Brinkmann Instruments, Westbury, N.Y.) and centrifuged. The concentrations of tissue inflammatory cytokines were measured using ELISA kits (TNF-α, IL-1β, and IL-6: Biosource International, California; and IL-8: Immuno-Biological Laboratories, Gunma, Japan) according to the manufacturer's recommendation.

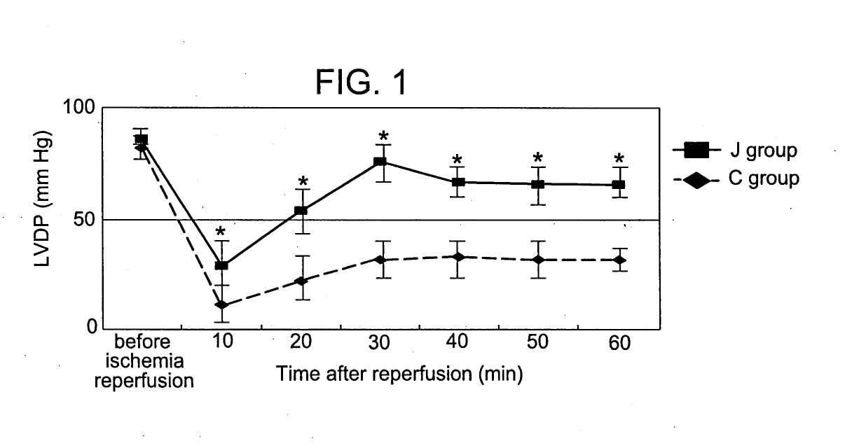

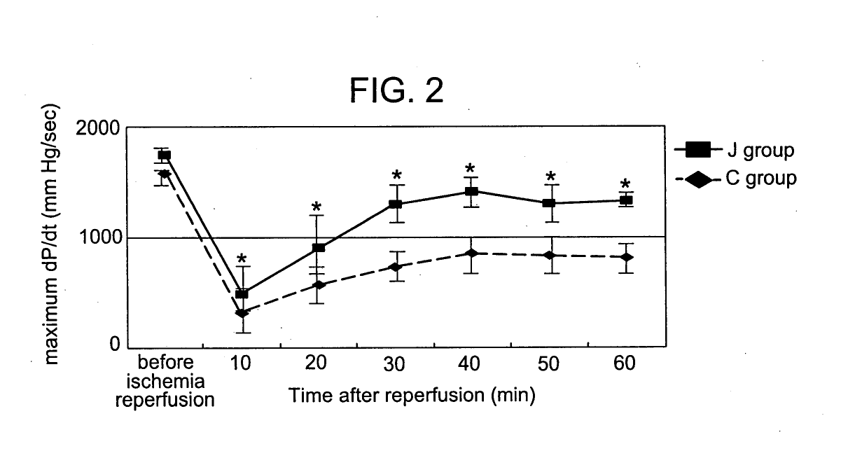

As depicted in FIGS. 1 and 2, there was no significant difference between the cardiac function (as measured by LVDP and maximum dP/dt) before ischemia reperfusion between the C and J groups. Similarly, there was no significant difference between the C and J groups in CF or HR before ischemia reperfusion.

There were significant differences between the two groups, however, following global ischemia and reperfusion. FIGS. 1 and 2 depict the cardiac function (as measured by LVDP and maximum dP/dt) after ischemia reperfusion between the C and J groups. For the following parameters, data are expressed as mean±standard error of the mean (SEM). The difference in the data on functional recoveries were determined with one-way repeated measure ANOVA. The difference in the data on CPK leakage and tissue cytokine concentrations were determined with unpaired Student's t test. A p value of less than 0.05 was considered statistically significant.

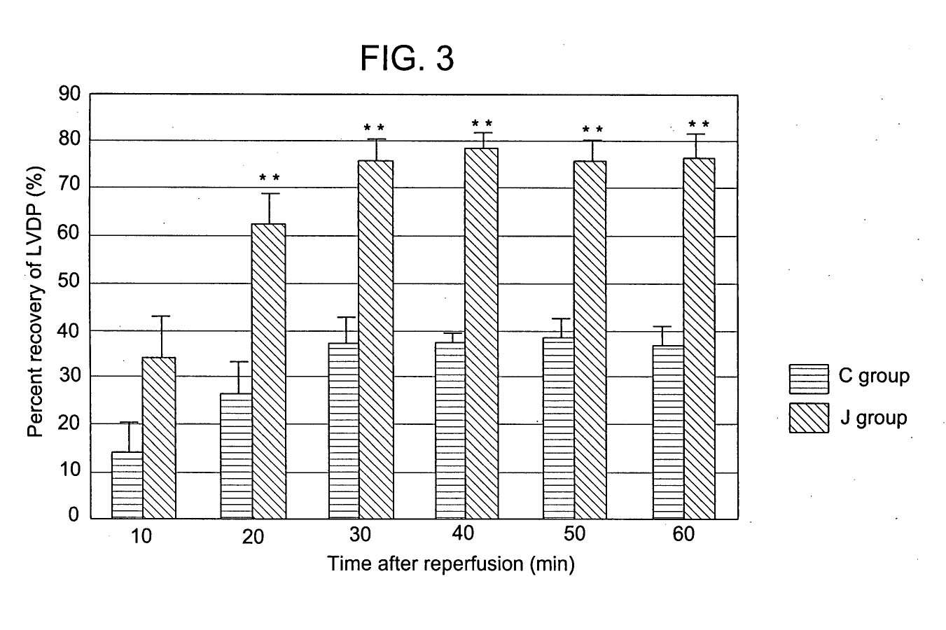

FIG. 3 details the percent recovery of LVDP after reperfusion. The isolated hearts from the C and J groups were subjected to 30 minutes of normothermic global ischemia followed by 60 minutes of reperfusion. Better recovery of LVDP after ischemia was shown in the J group than in the C group. In FIG. 3, data are expressed as a percentage of basal LVDP before ischemia. Significant improvements of the percent recovery of LVDP was observed in the J group at 20, 30, 40, 50, and 60 minutes after reperfusion. For example, the peak value of percent recovery of LVDP after reperfusion was 39±6% in the C group but was 79±5% in the J group (p<0.05).

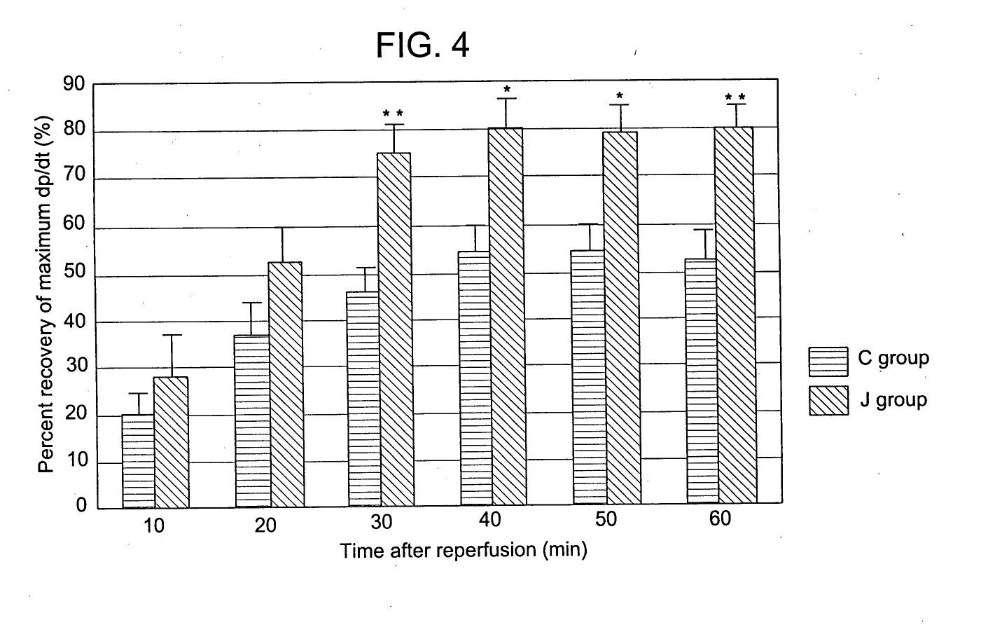

FIG. 4 details the percent recovery of maximum dP/dt after reperfusion. In comparison with the C group, a significant improvement of the percent recovery of max dP/dt was observed in the J group at 30, 40, 50, and 60 minutes after reperfusion. For example, the peak value of percent recovery of maximum dP/dt after reperfusion was 54±7% in the C group but was 80±4% in the J group (p<0.05).

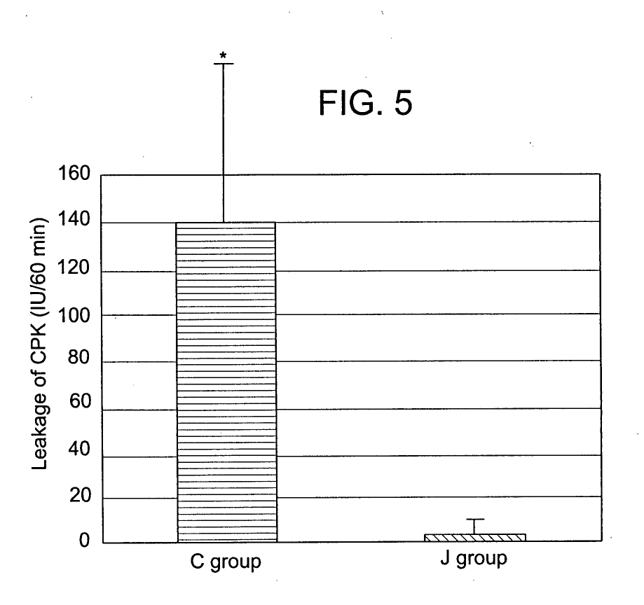

CPK leakage of the coronary effluent is detailed in FIG. 5. CPK activity was measured in the coronary effluent for 60 minutes after reperfusion. The J group significantly suppressed leakage of CPK from the cardiac myocytes as compared to the hearts of the C group. CPK leakage of the J group at 2.9±2.0 IU/60 minutes was significantly lower than that of the C group at 140±61 IU/60 minutes (p<0.05).

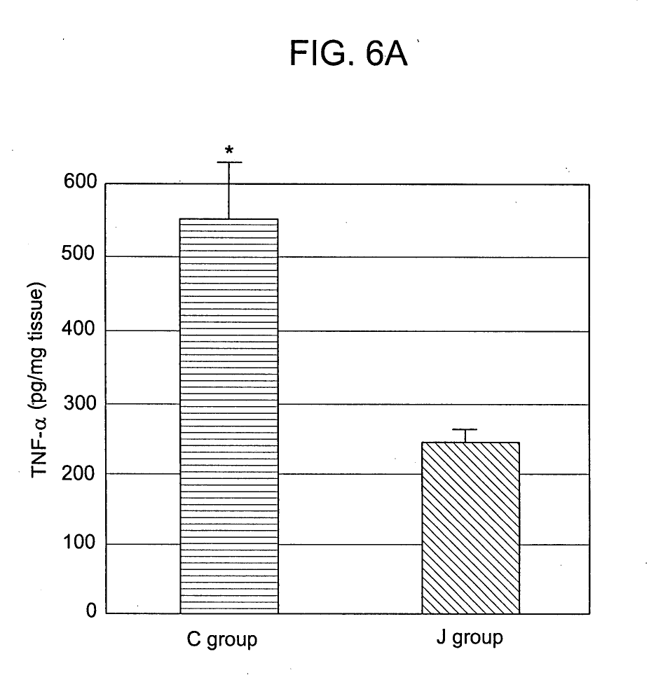

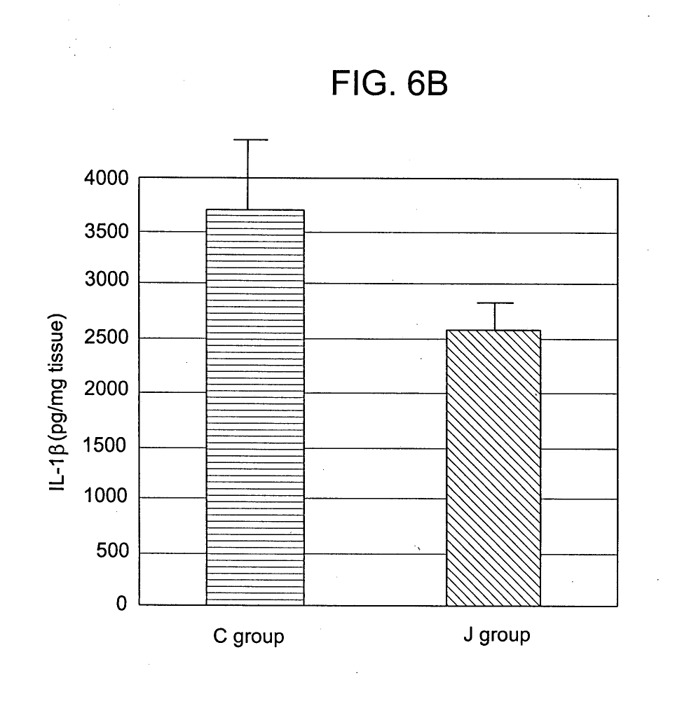

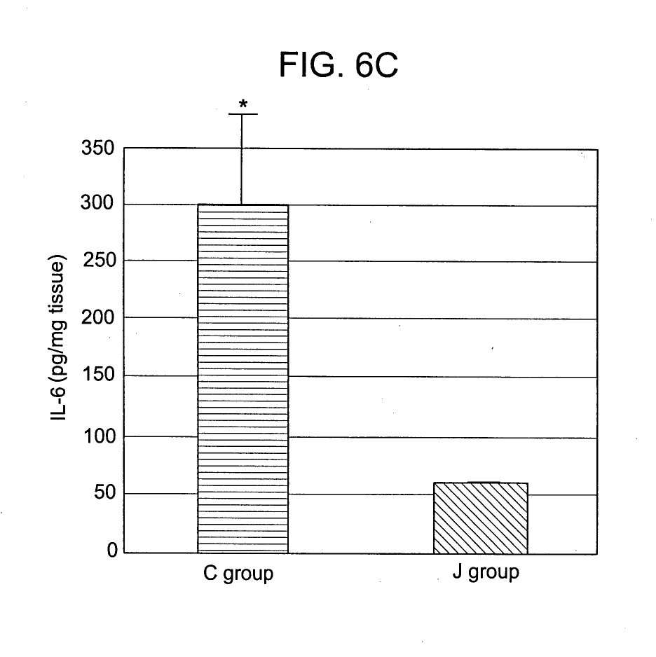

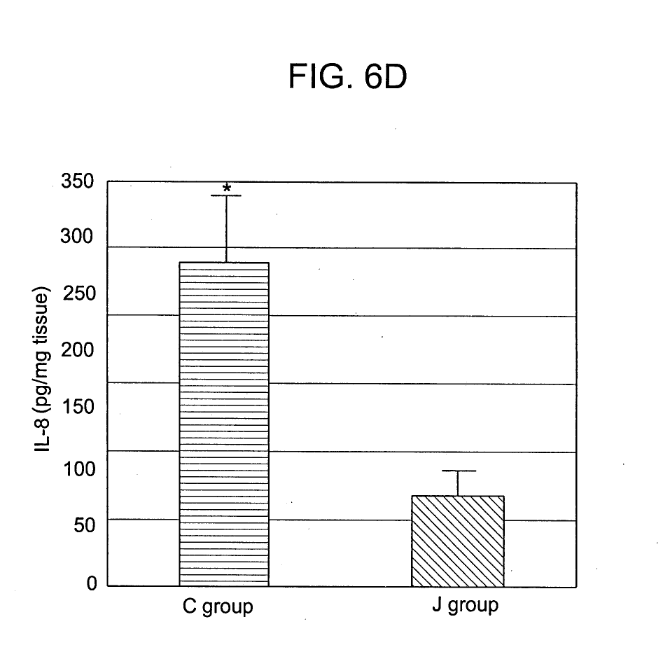

The amount of inflammatory cytokines in the tissues in J group also was significantly different from that of the C group. For example, as detailed in FIG. 6A, the tissue level of TNF-α in the myocardium after reperfusion was significantly lower in the J group as compared to the C group (240±30 pg/mg tissue versus 550±80 pg/mg tissue, respectively; p<0.05).

The tissue level of IL-6 similarly was significantly lower in the J group compared with the C group. As detailed in FIG. 6C, the tissue level of IL-6 in the myocardium after reperfusion was 60±7 pg/mg tissue versus 300±80 pg/mg tissue, respectively (p<0.05).

Moreover, the tissue level of IL-8 also was significantly lower in the J group compared to the C group. As detailed in FIG. 6D, the tissue level of IL-8 in the myocardium after reperfusion was 68±19 pg/mg tissue versus 240±40 pg/mg tissue, respectively (p<0.05).

While not statistically significantly different, the tissue level of IL-1β was lower in the J group compared to the C group. As detailed in FIG. 6B, the tissue level of IL-1β in the myocardium after reperfusion was 2600±200 pg/mg tissue versus 3700±700 pg/mg tissue, respectively.

Therefore, preconditioning with a cytokine production inhibitor (e.g., (−)-ethyl N-{3,5-dichloro-2-hydroxy-4-[2-(4-methylpiperazin-1-yl)ethoxy]benzoyl}-L-phenylalaninate dihydrochloride) results in treatment or prophylaxis of ischemia reperfusion injury as measured by (a) improved cardiac function (e.g., percent recovery of LVDP and percent recovery of maximum dP/dt), (b) decreased muscle injury (e.g., decreased CPK leakage), and (c) decreased pro-inflammatory cytokine production (e.g., production of IL-6, IL-8, and TNF-α).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for the treatment or prophylaxis of ischemia reperfusion injury in a subject in need thereof comprising administering a compound of Formula (I) to the subject: 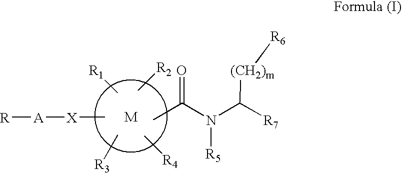
or a pharmaceutically acceptable acid addition salt thereof, wherein;
R is a piperazinyl optionally substituted by a lower alkyl, a piperidyl optionally substituted by a lower alkyl, or an amino,
wherein the amino is optionally substituted by a lower alkyl;
A is a linear alkylene;
X is an oxygen atom, a sulfur atom, —NH—, or —CH₂—;
M is an arylene;
R₁, R₂, R₃ and R₄ are the same or different and each is a hydrogen atom, provided at least one R₁, R₂, R₃, and R₄ is not a hydrogen atom, a hydroxy, a halogen atom, or —O—CO—R₁₁, wherein R₁₁ is a lower alkyl optionally substituted by a substituent selected from the group consisting of amino, acyloxy, and benzyloxycarbonyl, or phenyl optionally substituted by lower alkyl;
R₅ is a hydrogen atom;
m is 1;
R₆ is a phenyl; and
R₇ is —COO—R₁₂,
wherein R₁₂ is hydrogen atom, aralkyl, adamantyl, cyclohexylideneamino, cyclohexyl optionally substituted by lower alkyl, piperidyl optionally substituted by lower alky, or alkyl optionally substituted by a substituent selected from the group consisting of hydroxy, lower alkoxy, lower alkoxy lower alkoxy, lower alkoxycarbonyl, acyloxy, piperazinyl, and amino optionally substituted by lower alkyl.

2. The method of claim 1, wherein the compound is (−)-ethyl N-{3,5-dichloro-2-hydroxy-4-[2-(4-methylpiperazin-1-yl)ethoxy]benzoyl}-L-phenylalaninate or a pharmaceutically acceptable acid addition salt thereof.

3. The method of claim 1, wherein the compound is (−)-ethyl N-{3,5-dichloro-2-hydroxy-4-[2-(4-methylpiperazin-1-yl)ethoxy]benzoyl}-L-phenylalaninate dihydrochloride.

4. The method of claim 1, wherein ischemia reperfusion injury is cerebral, retinal, hepatic, renal, spinal cord, mesenteric, limb, intestinal, brain, myocardial, central nervous system, or lung ischemia reperfusion injury, or a combination thereof.

5. The method of claim 1, wherein the subject is a mammal.

6. The method of claim 5, wherein the mammal is a human.

7. The method of claim 1, wherein the production of at least one proinflammatory cytokine is inhibited.

8. The method of claim 7, wherein the cytokine is tumor necrosis factor (TNF)-α, interleukin (IL)-6, IL-8, or a combination thereof.

9. The method of claim 1, wherein, after the administration of the compound, the amount of creatine phosphokinase (CPK) released from a muscle after ischemia reperfusion is less than the amount of CPK released when the compound is not administered to the subject.

10. The method of claim 9, wherein the muscle is a cardiac muscle.

11. The method of claim 10, wherein, after the administration of the compound, the percent recovery of left ventricle developed pressure (LVDP) is greater by about 5% or more than the percent recovery of LVDP when the compound is not administered.

12. The method of claim 10, wherein, after the administration of the compound, the percent recovery of maximum rate of rise of left ventricular pressure (maximum dP/dt) is greater by about 5% or more than the percent recovery of maximum dP/dt when the compound is not administered.

13. The method of claim 1, wherein the compound is intraveneously administered to the subject.
